(19) United States
(12) Patent Application Publication
(10) Pub. No.: US 2008/0031883 A1
(43) Pub. Date: Feb. 7, 2008

(54) Condition-dependent, multiple target delivery system

(76) Inventors: Vladimir P. Torchilin, Charlestown, MA (US); Rishikesh Sawant, Boston, MA (US); Amit Kale, Boston, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/879,017

(22) Filed: Jul. 13, 2007

Related U.S. Application Data
(60) Provisional application No. 60/830,733, filed on Jul. 13, 2006.

(51) Int. Cl. A61K 31/50; A01N 1/00; A61K 31/70; A61K 38/00; A61K 39/395; A61K 49/00; A61P 43/00

(52) U.S. Cl. 424/152.1; 424/130.1; 424/9.1; 435/1.1; 514/2; 514/249; 514/44

(57) ABSTRACT

A condition-dependent, multiple target delivery system providing multifunctional, stimuli-sensitive pharmaceutical carriers is disclosed. The delivery system simultaneously carries on its surface various active moieties. The system is multifunctional and possesses the ability to switch on and switch off certain functions when necessary, for example, under the action of local stimuli characteristic of the target pathological zone (e.g., increased temperature or lowered pH values, which are characteristic of inflamed, ischemic and neoplastic tissues).

### CROSS REFERENCE TO RELATED APPLICATIONS

This application claims the benefit under 35 U.S.C. § 119(e) of U.S. Provisional Patent Application No. 60/830,733, filed on Jul. 13, 2006, the disclosure of which is incorporated by reference herein.

### STATEMENT REGARDING FEDERALLY SPONSORED RESEARCH OR DEVELOPMENT

Part of the work leading to this invention was carried out with United States Government support provided under a grant from the National Institutes of Health, Grants No. R01 LH55519 and R01 EB001961. Therefore, the U.S. Government has certain rights in this invention.

### BACKGROUND OF THE INVENTION

Intracellular transport of different biologically active molecules is one of the key problems in drug or diagnostic agent delivery in general. Ideally, a delivery system for biologically active molecules should be biodegradable and of small size, have good loading and prolonged circulation capacity and be able to specifically accumulate in the required organ or tissue, bypassing non-target areas. Once at the designated site, a delivery system should be able to penetrate inside the target cells, delivering its load (e.g., a small molecule drug, a nucleic acid, a diagnostic agent or a research reagent) intracellularly. Nanoparticular drug delivery systems, such as liposomes and micelles, are frequently used to increase the efficacy of drug and DNA delivery and targeting (Torchilin, 2005a; Torchilin, 2005b). In addition, within the past few years, it has been demonstrated that certain proteins and peptides (such as TAT peptide, or TATp) can enter cell cytoplasm directly and even target cell nuclei (Caron et al, 2001; Vives et al., 1997). These peptides have also been used successfully for the intracellular delivery of small drug molecules, large molecules (enzymes, DNA) and nanoparticulates (quantum dots, iron oxide nanoparticles, liposomes) (Torchilin et al., 2001b; Fawell et al., 1994; Rudolph et al., 2003; Santra et al., 2005; Schwarze et al., 1999; Torchilin et al., 2003a; Zhao et al., 2002). Yet multifunctional delivery systems that would combine these two effects, with specific targeting only to the site of need and intracellular delivery upon arrival, are clearly needed.

### BRIEF SUMMARY OF THE INVENTION

The condition-dependent, multiple target delivery system according to the invention addresses this need by providing multifunctional, stimuli-sensitive pharmaceutical carriers. The system of the invention simultaneously carries on its surface various active moieties. It is multifunctional and possesses the ability to switch on and switch off certain functions when necessary, for example, under the action of local stimuli characteristic of the target pathological zone (e.g., increased temperature or lowered pH values, which are characteristic of inflamed, ischemic and neoplastic tissues).

Different properties of the multifunctional drug delivery system of the invention are designed to be coordinated in a manner that is optimal for the intended use. For example, if the system is to be constructed to provide for target accumulation via enhanced permeability and retention and also for specific cell surface binding, allowing for its internalization by target cells, two requirements have to be met. First, the half-life of the carrier in the circulation system should be sufficient to fit the requirements for enhanced permeability, and second, the internalization of the delivery system by the target cells should proceed quickly enough not to allow for carrier degradation and loss in the interstitial space of the drug or other agent/reagent transported in the carrier.

The delivery system of the invention is constructed in such a way that a non-specific cell-penetrating function is shielded by a function providing for organ/tissue-specific delivery and/or by, e.g., sterically-protecting polymer molecules. Upon system accumulation in the target zone, the shielding agent, e.g., protecting polymer or antibody, which has been attached to the surface of the delivery particle via condition-dependent, stimuli-sensitive bonds, detaches under the action of local pathological conditions (e.g., abnormal pH or temperature) and exposes the previously hidden, second function, thus allowing for the subsequent delivery of the carrier and its cargo inside cells. Thus, the system of the invention minimizes the non-specific action of delivery systems, e.g., pharmaceutical systems, on normal tissues and cells while, at the same time, it provides for local delivery of, e.g., diagnostics, research reagents, drugs or nucleic acid only inside a target zone providing an appropriate stimulus that results in “deshielding.”

While such a system needs to be stable in the blood for a long time (on the order of hours) to allow for efficient target accumulation, it must lose its protective coat inside the target almost instantly to allow for fast internalization (on the order of minutes) to minimize the washing away of the released drug or DNA. Intracellular trafficking, distribution and fate of the carrier and its cargo can be additionally controlled by its charge and composition, which can drive it to the nuclear compartment or towards other cell organelles.

Thus, in one aspect, the invention is directed to a condition-dependent, multiple target delivery system that includes a polyfunctional carrier entity; a first class of targeting functionalities attached to the carrier entity and targeting a target zone; and a second class of targeting functionalities attached to the carrier entity, wherein the second class of targeting functionalities is shielded when the polyfunctional carrier entity is out of the target zone, but becomes exposed when the polyfunctional carrier entity is inside the target zone. The carrier entity in the delivery system may optionally be loaded with a molecule selected from the group consisting of a small molecule drug, a nucleic acid, a diagnostic agent and a research reagent, for delivery, e.g., to a patient, preferably a human patient, or for use in a tissue culture system.

Preferably, the polyfunctional carrier entity is selected from the group consisting of liposomes, micelles, polymeric particles, nanocapsules, niosomes and nanoparticles for delivery to a target zone in a patient including a tumor site, an infarct site, an infection site or an inflammation site. The first class of targeting functionalities preferably comprises an antibody, particularly a cardiac myosin-specific mAb 2G4, or nanoparticles. The second class of targeting functionalities is shielded, preferably, by a shielding construct comprising a first class targeting functionality attached to the carrier entity via a long-chain polymer spacer, preferably including a condition-dependent bond between the long-chain polymer spacer and the carrier entity. In another embodiment, the second class of targeting functionalities is shielded by a shielding construct comprising a sterically protective polymer, which is preferably poly (ethylene glycol), poly(vinyl alcohol) or poly(vinyl propionate), preferably including a condition-dependent bond to the carrier entity. The condition-dependent bonds preferably are cleavable under a condition at said target zone selected from the group consisting of a change in pH, a change in temperature, the presence of a redox agent, a change in oxygen content, enzyme activation, an increase in active oxygen content, an increase in free radical content and hypoxia. In preferred embodiments, the second class of targeting functionalities is a specific internalizable ligand, most preferably folate or transferrin, or a cell-penetrating peptide such as TATpeptide or polyarginine.

In another aspect, the invention is directed to a pharmaceutical composition including the delivery system according to the invention, wherein said carrier entity in the delivery system is loaded with a pharmaceutical agent. An effective amount of the pharmaceutical composition may be administered systemically or locally to a patient, particularly a human patient, for delivery of the pharmaceutical agent.

### BRIEF DESCRIPTION OF THE SEVERAL VIEWS OF THE DRAWINGS

Other features and advantages of the invention will be apparent from the following description of the preferred embodiments thereof and from the claims, taken in conjunction with the accompanying drawings, in which:

FIG. 1 is a schematic representation showing interaction of an exemplary multifunctional, pH-responsive delivery system according to the invention with a target cell. pH-dependent removal of protecting PEG chains or mAb-PEG moieties allows for the direct interaction of the cell-penetrating functionality with the cell membrane;

FIG. 2 is a schematic representation of the design of a multifunctional model delivery system including pH-cleavable PEG-Hz-PE (a), TATp (b), and monoclonal antibody (c) attached to the surface of a carrier entity via a pH-sensitive spacer;

FIG. 3 shows the steps in the preparation of a spacer function with a pH labile bond; FIG. 4 is a graph showing the results of HPLC analysis of the pH-sensitive mPEG₂₀₀₀-Hz-PE micelles after incubation at pH 8.0 (A) and after incubation at pH 5 (B) at room temperature;

FIG. 5 is a graph showing binding of anti-myosin mAb 2G4-PEG₂₀₀₀-Hz-PE-immunomicelles to a monolayer of dog cardiac myosin in comparison to the native mAb 2G4 at corresponding pH values; and

FIG. 6 is a bar graph showing binding of pH-sensitive biotin-micelles to NeutrAvidin columns after 15 min incubation at room temperature at pH 8.0 (a) and at pH 5.0 (b).

### DETAILED DESCRIPTION OF THE INVENTION

The multifunctional delivery system of the invention simultaneously carries on its surface various active moieties and possesses the ability to switch certain functions on and off when necessary, for example, under the action of local stimuli characteristic of the target pathological zone (e.g., increased temperature or lowered pH values, which are characteristic of inflamed, ischemic and neoplastic tissues). For example, organ or tissue accumulation (e.g., at sites of tumors, infarcts, infections, inflammations, etc.) can be achieved by passive targeting via the enhanced permeability and retention effect (Maeda et al., 2000; Palmer et al., 1984) or by antibody-mediated active targeting (Jaracz et al., 2005; Torchilin, 2004) while intracellular delivery can be mediated by certain, well-known internalizable ligands (e.g., folate, transferrin) (Gabizon et al., 2004; Widera et al., 2003) or cell-penetrating peptides such as TATpeptide or polyarginine (Gupta et al., 2005; Lochmann et al., 2004). It was shown that electrostatic interactions and hydrogen bonding lay behind the cell penetrating peptide-mediated direct transduction of small molecules (Mai et al., 2002; Vives et al., 2003), while the energy-dependent macropinocytosis is responsible for the cell penetrating peptide-mediated intracellular delivery of large molecules and nanoparticulates with their subsequent enhanced release from endosomes into the cell cytoplasm (Snyder et al., 2004; Wadia et al., 2004).

Different properties of the multifunctional delivery system of the invention are designed to be coordinated in a manner that is optimal for the intended use. For example, if the system is to be constructed to provide for target accumulation via enhanced permeability and retention and also for specific cell surface binding, allowing for its internalization by target cells, two requirements have to be met. First, the half-life of the carrier in the circulation should be sufficient to fit the requirements for enhanced permeability, and second, the internalization of the delivery system by the target cells should proceed quickly enough not to allow for carrier degradation and drug loss in the interstitial space.

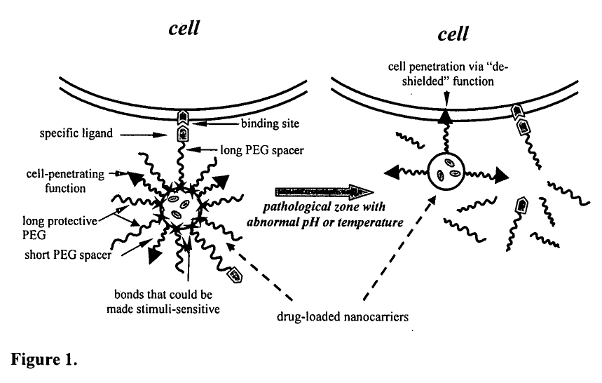

In concept, the delivery system according to the invention is constructed in such a way that during the first phase of system delivery, a non-specific cell-penetrating function is shielded by the function providing organ/tissue-specific delivery, and/or by sterically-protecting polymer molecules that are attached to the carrier via long-chain polymeric spacer, stimuli-degradable bonds. Upon accumulation in the target zone, the protecting releasable polymer, with or without as antibody or other targeting moiety attached to the surface of the delivery particle via stimuli-sensitive bonds, detaches under the action of local pathological conditions (e.g., abnormal pH or temperature) and exposes the previously hidden, second function, thus allowing for the subsequent delivery of the carrier and its cargo inside cells. While such a system should be stable in the blood for a long time (on the order of hours) to allow for efficient target accumulation, it must lose the protective coat inside the target almost instantly to allow for the fast internalization (on the order of minutes) to minimize the washing away of the released drug or DNA. The schematic pattern of such system is shown in FIG. 1. Intracellular trafficking, distribution and fate of the carrier and its cargo can be additionally controlled by its charge and composition, which can drive it to the nuclear compartment or towards other cell organelles.

In an exemplary model system, targeted long-circulating PEGylated liposomes and PEG-phosphatidylethanolamine (PEG-PE)-based micelles have been prepared, simultaneously carrying several functions. First, they have been made targeted by attaching the monoclonal antimyosin antibody 2G4 to their surface via the pNP-PEG-PE moieties. Second, these liposomes and micelles were additionally modified with biotin or TAT peptide (TATp) moieties attached to the surface of the nanocarrier by using biotin-PE or TATp-PE or TATp-shortPEG-PE derivatives. PEG-PE used for liposome surface modification or for micelle preparation was made degradable by inserting the pH-sensitive hydrazone bond between PEG and PE (PEG-Hz-PE). Under normal pH values, biotin and TATp functions on the surface of nanocarriers were “shielded” by longer protecting PEG chains (pH-degradable PEG₂₀₀₀-PE or PEG₅₀₀₀-PE) or by even more long pNP-PEG-PE moieties used to attach antibodies to the nanocarrier (non-pH-degradable PEG₃₄₀₀-PE or PEG₅₀₀₀-PE). At pH 7.5-8.0, both liposomes and micelles demonstrated high specific binding with 2G4 antibody substrate, myosin, but very limited binding on avidin column (biotin-containing nanocarriers) or internalization by NIH/3T3 or U-87 cells (TATp-containing nanocarriers). However, upon brief incubation (15-to-30 min) at lower pH values (pH 5.0-6.0) nanocarriers were losing their protective PEG shell because of acidic hydrolysis of PEG-Hz-PE and, in addition to their unchanged immune function acquired the ability to become strongly retained on avidin-column (biotin-containing nanocarriers) or effectively internalized by cells via TATp moieties (TATp-containing nanocarriers).

Exemplary carrier entities include liposomes, micelles, polymeric particles, nanocapsules, niosomes and nanoparticles for delivery of an agent to such exemplary targets as tumors, coronary infarcts, infection sites and general inflammation sites. “De-shielding” of the secondary targeting functionalities through rupture of the stimulus degradable bond can occur under a variety of conditions, such as, but not limited to, a change in pH, a change in temperature, the presence of a redox agent (such as GSH (—SH), an increase in oxygenation levels (P_{O2}), enzyme activation (such as proteolysis or metalloproteolysis), an increase in active oxygen content (such as superoxide or singlet oxygen) or free radical content, or hypoxia. Exemplary pH-sensitive linkages include hydrazone as described herein, cis-aconityls (Shen et al., 1981; Ogden et al., 1989), electron-rich trityls (Patel et al., 1996); polyketals (Heffernan et al., 2005), acetals (Gillies et al., 2005; Gillies et al., 2004), vinyl ethers (Gumusderelioglu et al., 2005; Shin et al., 2003), poly(ortho-esters) (Toncheva et al., 2003), thiopropionates (Oishi et al., 2005), and N-ethoxybenzylimidazoles (Kong et al., 2007). Also useful are peptide bonds sensitive to the action of local proteases, e.g., metalloproteases, (Rijken et al., 2007). Examples of shielding functionalities for the surface of micelles include a pH-sensitive polymer coat (Lee et al., 2005). TATp-function attached to PEG can be shielded by another longer spacer pH-sensitive block polymer that exposes TATp when incubated in acidic pH (Sethuraman et al., 2007).

The following examples are presented to illustrate the advantages of the present invention and to assist one of ordinary skill in making and using the same. These examples are not intended in any way otherwise to limit the scope of the disclosure.

### EXAMPLE

### Model Preparation of a Multifunctional Delivery System

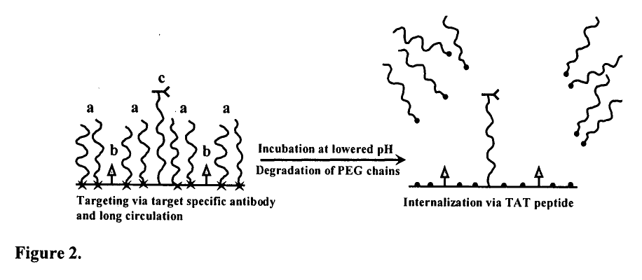

The particular design of the model system used is presented in FIG. 2. Referring to FIG. 2, an exemplary carrier (e.g., liposome or micelle) bears on its surface 20 (1) a “hidden” function 22 (biotin and TATpeptide moieties were used in this example) inserted into the liposome membrane or micelle core via modification with PE moiety; (2) protecting PEG chains 24 (e.g., PEG₂₀₀₀) attached to the surface via a pH-cleavable bond 26; and (3) specific antibody 28 attached to the surface via non-cleavable, long PEG spacers (PEG₃₄₀₀). In some experiments with liposomes, cleavable PEG₅₀₀₀-Hz-PE and non-cleavable TATp-PEG₂₀₀₀-PE conjugates have been used.

If the model system functions as expected, the delivery system will demonstrate specific targeted properties (via antibody-mediated recognition) at both normal (7.5-8.0) and acidic (5.0-6.0) pH values; however, the incubation of the model construct at lowered pH should eliminate (detach) protecting PEG chains and de-shield the second function. In other words, after exposure to the lowered pH, in addition to the immune recognition, the delivery system should acquire the ability to bind with an avidin column if the second, “hidden” function is biotin, or to demonstrate better internalization by the target cells if the second, “hidden” function is TATp. Cardiac myosin-specific monoclonal 2G4 antibody (mAb 2G4) (Liang et al., 2004) was used as the targeting antibody. The coupling of mAb 2G4 and biotin or TATp to the carrier entity surface was performed using the reactive derivative of poly(ethylene glycol)-phosphatidyl ethanolamine conjugate (PEG-PE) activated at the free PEG terminus with a p-nitrophenylcarbonyl (pNP) group (pNP-PEG-PE) according to a protocol developed earlier (Torchilin et al., 2001a).

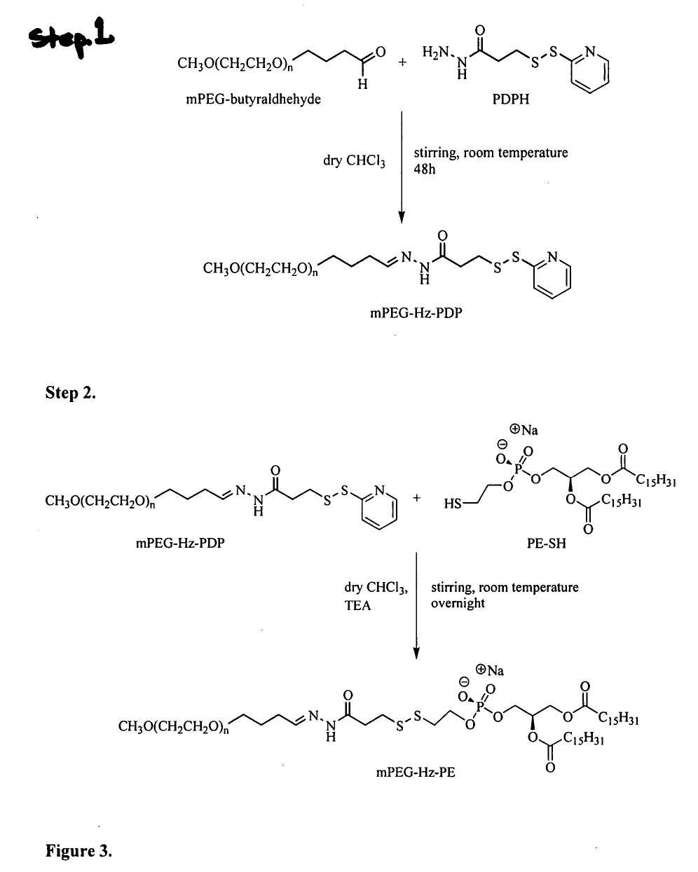

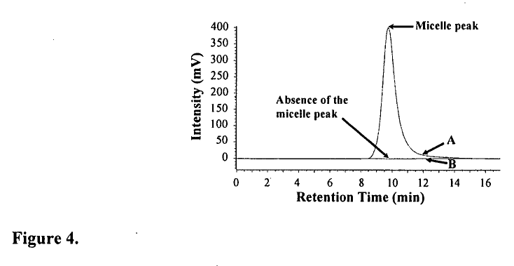

The steps for the synthesis of PEG-PE conjugated via the pH-cleavable hydrazone group (PEG-Hz-PE) are shown in the FIG. 3. The synthesis was carried out in two steps. The first step involves the conjugation of 3-(2-pyridyldithio) propionyl hydrazide (PDPH) to mPEG₂₀₀₀-CHO. The hydrazide group in PDPH reacts with the aldehyde group of mPEG₂₀₀₀-CHO to form the acidic-pH-labile hydrazone bond. Since this bond is vulnerable to hydrolysis, efforts were taken to carry out the reaction in anhydrous conditions. Use of PDPH as the cross linker not only offers the advantage of forming the hydrazone bond but also introduces pyridyldisulfanyl groups for subsequent conjugation of PEG to the thiol component of 1,2-dipalmitoyl-sn-glycero-3-phosphothioethanol (PE-SH) in the second step of the synthesis. The PE-SH moiety in the conjugate serves as a hydrophobic anchor to assure its association with the lipid bilayer of liposome or the hydrophobic core of micelles. All steps of the reaction were followed on TLC to confirm the progress of the reaction. The structure of the final conjugate, mPEG₂₀₀₀-phospatidyl ethanolamine hydrazone (mPEG₂₀₀₀-Hz-PE) was confirmed by the proton NMR characterization.

The stability of the conjugate and the kinetics of its degradation were analyzed by size-exclusion HPLC, following the area under the micelle peak on the chromatogram after conjugate incubation for different time intervals at different pH values (the conjugate spontaneously forms micelles in aqueous solutions similar to “normal” PEG-PE conjugate). Rhodamine-PE was incorporated into the micelles as a fluorescent tag, and the sample was monitored using fluorescence detection with excitation at 550 nm and emission at 590 nm. As a typical example, HPLC results for mPEG₂₀₀₀-Hz-PE are shown in FIG. 4; after the appropriate pH treatment, incubation at pH 5.0, the peak at retention time 9.7 min (the micelle peak), observed in the presence of intact micelles at pH 8.0, disappears. The disappearance of this peak is indicative of the destruction of the micelle structure due to the loss of PEG corona, while non-pH-sensitive micelles produce the peak at both pH values. Table I shows the representative data for the degradation kinetics of PEG₂₀₀₀-Hz-DPPE micelles at different pH values. It is clearly seen from these results that, although such micelles are quite stable at high pH value (8 and above), they disintegrate within a few minutes at pH 5.0. This result was also confirmed by TLC, when two different spots of PEG and PE were observed after a few minutes of incubation at pH 5.0 with no spot of PEG-PE seen after the incubation. This result illustrates the viability of condition-dependent bonds.

TABLE I
PEG₂₀₀₀-Hz-PE micelle stability at different
pH values (as percent of remaining micelles)

pH value

| Incubation Time | 5.0 | 7.0 | 8.0 | 10.0 |
|---|---|---|---|---|
| 20 min | 3 | 56 | 94 | 99 |
| 40 min | 2.5 | 28 | 62 | 99 |
| 60 min | 2 | 10 | 53 | 99 |

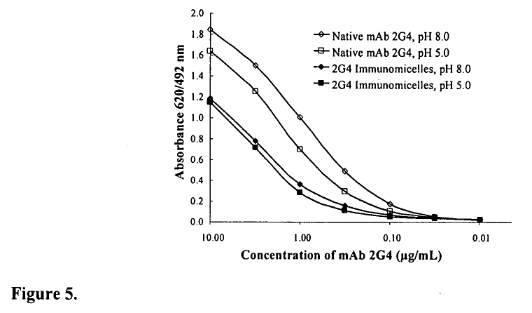

As a multifunctional delivery system, fully assembled 2G4 antibody-bearing model carrier entities demonstrated clear immunoreactivity towards the antigen, the monolayer of dog cardiac myosin in the standard ELISA test, at both tested pH values, 8.0 and 5.0, as can be seen in FIG. 5. Although, one can observe some affinity decrease for the antibodies modified with the pNP-PEG-PE anchor and incorporated into the micelle structure (the same pattern is observed for immunoliposomes), this decrease is more apparent than real, since not all delivery system-attached antibodies, even remaining active, can interact with the substrate because of their steric orientation of the carrier entity surface, and, as was shown earlier, this restriction is well compensated for by the multipoint interaction of antibody-modified carrier entities with the target (Klibanov et al., 1985; Lukyanov et al., 2004). Thus, the systems prepared are immunologically active at both chosen pH values. Control preparations bearing a non-specific IgG did not show any binding with myosin at any pH.

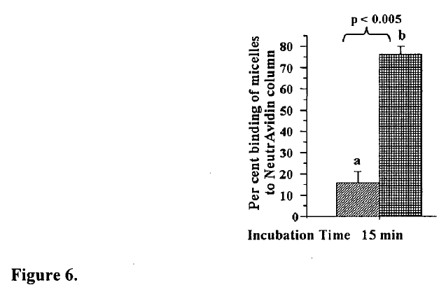

The avidin-biotin complexation was used initially as an easy-to-handle test system to follow the shielding and de-shielding of the second hidden function in pH-sensitive model functionalized delivery systems. Therefore, liposomes and micelles containing 5% mol of the biotin-PE in addition to 2G4-PEG₃₄₀₀-PE and pH-sensitive PEG₂₀₀₀-Hz-PE were prepared and labeled with rhodamine-PE, and their ability to interact with avidin (NeutrAvidin affinity column) was investigated at pH 8.0 and after a brief (15 min) exposure at the lowered pH of 5.0. It was found that, although biotin-containing 2G4-antibody labeled carrier entities have demonstrated identical immunoreactivity at both pH values, their ability to bind with avidin was dramatically different at pH 8.0 as compared to after a 15 min incubation at pH 5.0, which was expected to cleave away a substantial portion of the shielding PEG₂₀₀₀ micelle corona (or liposome coating). The data in FIG. 6 (for micellar carrier entities) clearly show that while at pH 8.0 only about 15% of micelles were retained by the avidin column, after 15 min incubation at pH 5.0, about 75% of micelles were retained (the degree of the binding was estimated following the decrease in the sample rhodamine fluorescence at 550/590 nm after passing through the avidin column). This result clearly confirms that the elimination of the pH-cleavable PEG coat de-shields the hidden biotin function and allows for more biotin moieties to interact with avidin on the column.

For cell culture experiments, a rhodamine-labeled delivery system similar to those described above, but containing TATp moieties attached to the surface instead of biotin groups, was used. Delivery system internalization by various cells (non-targets for the 2G4 antibody) was inventigated at pH 8.0 and after the brief (20-30 min) exposure at pH 5.0. It was found that TATp-containing carrier entities also demonstrate a dramatically different ability to interact with cells at pH 8.0 and after the incubation at pH 5.0. While cleavable PEG-PE-based TATp-containing micelles kept at pH 8.0 show only marginal association with NIH-3T3 murine fibroblasts, the same micelles pre-incubated for 30 min at pH 5.0 demonstrated dramatically enhanced association with the cells (higher fluorescence), i.e. better accessibility of TATp moieties for cell interaction. In case of TAT-bearing liposomes, the incorporation of 9% mol of PEG-PE strongly diminished TATp-uptake of liposomes, and the incorporation of 18% mol of PEG-PE completely eliminated it. However, when pH-degradable PEG-PE was used, a 20 min preincubation of both preparations at pH 5.0 significantly increased the association of both preparations with cells, bringing the cell binding of the liposomes with 9% mol PEG almost back to the level of PEG-free TATp-liposomes, and significantly improving the cell binding of the TATp-liposomes with 18% mol of the initial PEG. These results clearly confirm that the elimination of the pH-cleavable PEG coat de-shields the hidden TATp function and allows for better association of functionalized carrier entity with the cells.

Materials and Methods

Materials

Cell lines, mouse fibroblast NIH 3T3 and human astrocytoma U-87 MG, were purchased from the American Type Culture Collection (Manassas, Va.). All cell culture media, DMEM, and RPMI 1640, heat-inactivated fetal bovine serum (FBS), and concentrated solutions of sodium pyruvate and penicillin/streptomycin stock solutions were purchased from Cellgro® (Herndon, Va.). TAT-peptide (11-mer: TyrGlyArgLysLysArgArgGlnArgArgArg; molecular mass, 1,560 Da; three reactive amino groups) was prepared by Research Genetics (Huntsville, Ala.). Monoclonal antibody (mAb) 2G4 was produced and purified by the inventors [Do we have a reference?]. pNP-PEG₃₄₀₀-PE was synthesized and purified according to an established method (Torchilin et al., 2001a). Egg phosphatidylcholine (PC), cholesterol, 1,2-Distearoyl-sn-Glycero-3-Phosphoethanolamine-N-[Methoxy(Polyethylene glycol)-750] (Ammonium Salt) (mPEG₇₅₀-PE), 1,2-Distearoyl-sn-Glycero-3-Phosphoethanolamine-N-[Methoxy (Polyethylene glycol)-2000] (Ammonium Salt) (mPEG₂₀₀₀-PE), 1,2-Dipalmitoyl-sn-Glycero-3-Phosphoethanolamine-N-(Cap Biotinyl) (Sodium Salt) (biotin-PE) 1,2-Dipalmitoyl-sn-Glycero-3-Phosphothioethanol (Sodium Salt) (PE-SH) and 1,2-Dioleoyl-sn-Glycero-3-Phosphoethanolamine-N-(Lissamine Rhodamine B Sulfonyl) (Ammonium Salt) (Rh—PE) were obtained from Avanti Polar Lipids, Inc. (Alabaster, Ala.). mPEG₂₀₀₀-butyraldehyde (mPEG₂₀₀₀-CHO) and mPEG₅₀₀₀-butyraldehyde (mPEG₅₀₀₀-CHO) was obtained from Nektar™ (Huntsville, Ala.). Control bovine antibody IgG was obtained from Serologicals Proteins, Inc. (Kankakee, Ill.). 3-(2-Pyridyldithio) propionyl hydrazide (PDPH) and Immobilized NeutrAvidin™ Protein was purchased from Pierce Biotechnology, Inc. (Rockford, Ill.). Bovine serum albumin and all other chemicals and buffer solution components were from Sigma (St. Louis, Mo.) and were of analytical grade.

Methods

Synthesis of pH-Cleavable mPEG₂₀₀₀-hydrazone-phospatidyl ethanolamine (mPEG₂₀₀₀-Hz-PE).

Developing the method of coupling oxidized antibody to the PEG terminus through a hydrazone bond as suggested by Hansen et al (Hansen et al., 1995), we devised our own scheme of conjugate reaction for synthesis of pH-cleavable PEG-PE. The reaction was performed in two steps: first, the activation of mPEG₂₀₀₀-CHO with PDPH, and second, the conjugation of 1,2-dipalmitoyl-sn-glycero-3-phosphothioethanol (Na salt) (PE-SH) to activated mPEG-CHO.

Referring to FIG. 3, for step 1 of the synthesis, 150 mg (64 μmole) of mPEG₂₀₀₀-CHO was dissolved in dry chloroform containing 3.5 molar excess of 3-(2-pyridyldithio) propionyl hydrazide (PDPH) to obtain 50 mg/ml solution of mPEG₂₀₀₀-CHO. The mixture was incubated for 48 h at room temperature with stirring under argon. TLC (CHCl₃:CH₃OH:H₂O—80:20:2) revealed that the reaction was complete. The starting material mPEG₂₀₀₀-CHO did not absorb UV and was positive to Dragendorff spray, while PDPH absorbed UV and was negative for Dragendorff spray. The product, mPEG₂₀₀₀-Hz-PDP, absorbed UV and was positive to Dragendorff spray. Organic solvents were then removed using a rotary evaporator. mPEG₂₀₀₀-Hz-PDP was then dissolved in deionized water (adjusted to pH 10-11 using 1 M NaOH) and purified from unreacted PDPH using Sepharose G25 column and deionized water (adjusted to pH 10.5 using 1 M NaOH). Pooled fractions containing mPEG₂₀₀₀-Hz-PDP (dragendorff and UV positive) were freeze-dried.

For step 2 of the synthesis, 20 mg (26 μmole) of 1,2-Dipalmitoyl-sn-Glycero-3-Phosphothioethanol (Sodium Salt) (PE-SH) was dissolved in dry chloroform containing 1.5 molar excess of mPEG₂₀₀₀-Hz-PDP, to get a 10 mg/mL solution of PE-SH. The solution was supplemented with 15 μL (approx. 3-fold molar excess over PEG) of triethylamine (TEA). The sample was incubated overnight at room temperature with stirring under argon. TLC (CHCl₃:CH₃OH:H₂O—80:20:2) revealed that the reaction was complete. The starting material mPEG₂₀₀₀-Hz-PDP was positive to dragendorff spray and negative for molybdenum blue, while PE-SH was positive to molybdenum blue and negative for Dragendorff spray. The product, mPEG₂₀₀₀-Hz-PE, was positive to both dragendorff spray and molybdenum blue. The organic solvents were then removed using a rotary evaporator. The mPEG₂₀₀₀-Hz-PE micelles were formed in deionized water (adjusted to pH 10.5 using 1 M NaOH) by vortexing. The micelles were separated from the unbound PEG and released pyridine-2-thione on CL-4B column using deionized water (adjusted to pH 10.5 using 1 M NaOH) as an eluent. Pooled fractions containing mPEG₂₀₀₀-Hz-PE were freeze-dried, and was extracted with chloroform. mPEG₂₀₀₀-Hz-PE was stored as 10 mg/mL chloroform solution at −80° C. under argon until further use. mPEG₅₀₀₀-Hz-PE used in some experiments was synthesized in the same way starting with mPEG₅₀₀₀-CHO. ¹H NMR (500 MHz, CDCl₃) δ (ppm) for mPEG₂₀₀₀-Hz-PE: 0.87 (t, CH₃ of lipid, 6H), 1.27 (b, s, CH₂, ≈56H), 2.29 (t, OCOCH₂, 4H), 2.40 (t, COCH₂CH₂S, 2H), 2.45 (t, SCH₂CH₂O, 2H), 2.5 (t, COCH₂CH₂S, 2H), 2.59 (m, CH₂CH═N, 2H), 3.1 (t, CH₂CH, 2H), 3.39 (s, OCH₃ of PEG, 3H) and 3.5 (bm, PEG, ≈184H). Thus, there was a clear indication for the presence of expected conjugate.

Acidic pH Cleavability of mPEG₂₀₀₀-Hz-PE.

TLC analysis. TLC-verified degradation of the polymer conjugates after pH treatment and spots corresponding to plain PEG and plain PE were observed after incubation of the polymers at pH 5.0 for 15 minutes at 37° C.

HPLC analysis. Micelles of mPEG₂₀₀₀-Hz-PE were prepared containing 1 mol % of rhodamine-PE as fluorescent marker as follows. Lipid film was prepared by mixing chloroform solutions of both the lipids in a round bottom flask and then removing chloroform using rotary evaporator. To ensure complete removal of any traces of chloroform further drying was done using lyophilizer. Appropriate volume of pH 8.5 phosphate buffer (100 mM phosphate, 150 mM sodium chloride) was added and vortexed for 2 min to form 0.5 mM solution of mPEG₂₀₀₀-Hz-PE micelles. Sample was then divided into equal volumes and treated for different pH incubation. For pH 7.4 treatment, a 50 μL aliquot of first half of the micelle formulation was applied, as is, to Shodex KW-804 size exclusion column at regular intervals using pH 7.4 Phosphate buffer (100 mM phosphate, 150 mM sodium sulfate) as eluent and run at 1.0 ml/min. Both UV (from 200 to 400 nm) and fluorescence (550/590) was used to monitor the micelles. To the second half of the micelle formulation appropriate volume of 1N HCl was added to get a final pH of 5.0; aliquots of which were then analyzed as above at different intervals. As a control, micelles of mPEG₂₀₀₀-PE (non-pH-sensitive micelles) were prepared and analyzed after treatment at both the pH values as above.

Kinetics of the pH-Dependent Degradation of mPEG₂₀₀₀-Hz-PE.

The degradation of the micelles spontaneously formed by mPEG₂₀₀₀-Hz-PE under the action of the acidic pH was studied by following the presence or absence of micelle over the period of time in buffer solutions of different pHs (i.e. pH values 6.0, 7.0, 8.0 and 10). Rh—PE-labeled micelles of mPEG₂₀₀₀-Hz-PE conjugate were prepared in phosphate buffer (10 mM phosphate, 150 mM NaCl) solutions of different pH values. The pH of the solution was adjusted using appropriate amounts of either 1N HCl or NaOH. 50 μl aliquots were sampled out at different time intervals for size exclusion chromatographic analysis in 100 mM phosphate buffer (pH 7.0) containing 150 mM sodium sulphate using fluorescence detector (EX: 550 nm, EM: 590 nm). The area under micelle peak (mean retention time: 9.35 min) was determined for each chromatogram.

Preparation of pH-Sensitive Drug Delivery Systems According to the Invention.

Micelles. For micelle preparations, a mixture of mPEG₇₅₀-PE, pH-sensitive mPEG₂₀₀₀-Hz-PE, biotin-PE (or TATp-PE), and Rh—PE at molar ratio of 40:54:5:1 was prepared in chloroform. Chloroform was removed on a rotary evaporator followed by freeze-drying on a Freezone 4.5 (Labconco, Kansas City, Mo.). The film was hydrated with PBS, pH 8.0 (10 mM phosphate, 150 mM sodium chloride) at room temperature and vortexed for 5 min. Micelle size was controlled by using a Coulter N4 Plus submicron particle analyzer.

Liposomes. For liposome preparations, a mixture of phosphatidylcholine and cholesterol in 6:3 molar ratio and with the addition of various quantities (up to 18% mol) of PEG₅₀₀₀-Hz-PE or mPEG₅₀₀₀-PE was prepared in chloroform. When required, the composition for liposome preparation was supplemented with 0.5 to 1% mol of TATp-PEG₂₀₀₀-PE (prepared as described earlier in (Torchilin et al., 2001b)) and with 0.5% mol of Rh—PE (for the fluorescent labeling). Chloroform was removed on a rotary evaporator followed by freeze-drying on a Freezone 4.5 (Labconco, Kansas City, Mo.). The film obtained was hydrated with HBS buffer (pH 8.0) at room temperature for 5 min. The lipid dispersion was extruded 20 times through polycarbonate filters (pore size 200 nm) by using a Micro extruder (Avanti). Vesicle size was controlled by using a Coulter N4 Plus submicron particle analyzer.

Preparation of Immunocarriers.

First, mAb 2G4 or nonspecific control bovine IgG was conjugated to pNP-PEG₃₄₀₀-PE as in (Torchilin et al., 2001a) with some modifications. Briefly, pNP-PEG₃₄₀₀-PE and mPEG₇₅₀-PE was dried in a rotary evaporator and freeze-dryer to form a thin film. The film was hydrated with 5 mM citrate buffered saline, pH 5.0, and vortexed. Antibody solution was prepared in 50 mM tris-buffered saline, pH 8.7 and incubated with a 10-fold molar excess of pNP-PEG₃₄₀₀-PE for 24 h at 4° C. to allow the attachment of the antibody to the activated PEG terminus with the simultaneous hydrolysis of non-reacted pNP groups, thus forming the antibody-micelle solution. Then, the required aliquot of this solution was added to liposome or micelles prepared as described above and incubated for about an hour to allow for the quantitative incorporation of the modified antibody into the appropriate DDS (Torchilin et al., 2003b).

Assays

An ELISA assay (indirect, using an enzyme-tagged secondary Ab) was performed to show the ability of the pH-sensitive immunocarriers to recognize the target antigen at different pH values (pH 8.0 and 5.0).

First, ELISA plates were coated with 50 μl of 10 μg/ml cardiac myosin and incubated overnight at 4° C. Then, each well was washed three times with 200 μl of TBST (TBS containing 0.05% w/v Tween-20), and incubated with 50 μl of serial dilutions of 2G4 antibody (or non-specific IgG) in TBST-Casein (TBST with 2 mg/mL casein) for 1 h at RT. After incubation, the wells were washed as before and incubated with 50 μl/well of 1:5000 dilution of goat anti-mouse IgG peroxidase conjugate (ICN Biomedicals, Inc., Aurora, Ohio) in TBST-Casein for 1 h at RT. The wells were again washed as before, and each well was incubated with 100 μl of enhanced Kblue® TMB peroxidase substrate (Neogen Corporation, Lexington, Ky.) for 15 min. The microplate was read at a dual wavelength of 620 nm with the reference filter at 492 nm using a Labsystems Multiskan MCC/340 microplate reader installed with GENESIS-LITE windows based microplate software.

Biotin-Avidin Binding

To test the binding of biotin-bearing Rh—PE-labeled DDS before and after incubation at lowered pH values, the corresponding samples were kept for 15 min at pH 8.0 or pH 5.0 and then applied onto the Immobilized NeutrAvidin™ protein column. The degree of the retention of the corresponding preparation on the column was estimated following the decrease in the sample rhodamine fluorescence at 550/590 nm after passing through the NeutrAvidin™ column

Interaction of TATp-Containing pH-Sensitive Systems According to the Invention with Cells.

For experiments with the micelles, NIH 3T3 cells (fibroblasts) have been chosen. After the initial passage in tissue culture flasks, NIH 3T3 cells were grown on coverslips in 6-well tissue culture plates (100,000 cells per well) in DMEM with 10% BSA. After 48 h the plates were washed twice with PBS, pH 7.4, and then treated with various Rh—PE-labeled micelle samples (without and with pre-incubation for 15 min at pH 5.0) in serum-free medium (2 ml/well, 30 mg total PEG-PE/ml). After a 1 h incubation period, the media were removed and the plates washed with serum-free medium three times. Individual coverslips were mounted cell-side down onto fresh glass slides with PBS. Cells were viewed with a Nikon Eclipse E400 microscope under bright light, or under epifluorescence with rhodamine/TRITC.

For experiments with the liposomes, U-87 MG cells (astrocytoma) have been chosen. After the initial passage in tissue culture flasks, U-87 MG cells were grown on coverslips in 6-well tissue culture plates (20,000 cells per well) in DMEM with 10% BSA. After 48 h the plates were washed twice with PBS, pH 7.4, and then treated with various Rh—PE-labeled liposome samples (with and without pre-incubation for 20 min at pH 5.0) in serum-free medium (2 ml/well, 30 mg total lipid/ml). After a 1 h incubation period, the media were removed and the plates washed with serum-free medium three times. Individual coverslips were mounted cell-side down onto fresh glass slides with PBS. Cells were viewed with a Nikon Eclipse E400 microscope under bright light, or under epifluorescence with rhodamine/TRITC.

Use

The compositions of the invention may be administered orally, topically, or parenterally (e.g., intranasally, subcutaneously, intramuscularly, intravenously, or intra-arterially) by routine methods in pharmaceutically acceptable inert carrier substances. For example, the compositions of the invention may be administered in a sustained release formulation using a biodegradable biocompatible polymer, or by on-site delivery using micelles, gels or liposomes. The compositions of the invention can be administered in a dosage of 0.25 μg/kg/day to 5 mg/kg/day, and preferably 1 μg/kg/day to 500 μg/kg/day. The specific dosage will be dependent on the specific compound carried by the delivery system according to the invention. Optimal dosage and modes of administration can readily be determined by conventional protocols.

### REFERENCES

Caron, N. J., Torrente, Y., Camirand, G., Bujold, M., Chapdelaine, P., Leriche, K., Bresolin, N., and Tremblay, J. P. (2001) Intracellular delivery of a Tat-eGFP fusion protein into muscle cells. Mol Ther 3, 310-8.
Fawell, S., Seery, J., Daikh, Y., Moore, C., Chen, L. L., Pepinsky, B., and Barsoum, J. (1994) Tat-mediated delivery of heterologous proteins into cells. Proc Natl Acad Sci U S A 91, 664-8.
Gabizon, A., Shmeeda, H., Horowitz, A. T., and Zalipsky, S. (2004) Tumor cell targeting of liposome-entrapped drugs with phospholipid-anchored folic acid-PEG conjugates. Adv Drug Deliv Rev 56, 1177-92.
Gupta, B., Levchenko, T. S., and Torchilin, V. P. (2005) Intracellular delivery of large molecules and small particles by cell-penetrating proteins and peptides. Adv Drug Deliv Rev 57, 637-51.
Hansen, C. B., Kao, G. Y., Moase, E. H., Zalipsky, S., and Allen, T. M. (1995) Attachment of antibodies to sterically stabilized liposomes: evaluation, comparison and optimization of coupling procedures. Biochim Biophys Acta 1239, 133-44.
Jaracz, S., Chen, J., Kuznetsova, L. V., and Ojima, I. (2005) Recent advances in tumor-targeting anticancer drug conjugates. Bioorg Med Chem 13, 5043-54.
Klibanov, A. L., Muzykantov, V. R., Ivanov, N. N., and Torchilin, V. P. (1985) Evaluation of quantitative parameters of the interaction of antibody-bearing liposomes with target antigens. Anal Biochem 150, 251-7.
Liang, W., Levchenko, T., Khaw, B. A., and Torchilin, V. (2004) ATP-containing immunoliposomes specific for cardiac myosin. Curr Drug Deliv 1, 1-7.
Lochmann, D., Jauk, E., and Zimmer, A. (2004) Drug delivery of oligonucleotides by peptides. Eur J Pharm Biopharm 58, 237-51.
Lukyanov, A. N., Elbayoumi, T. A., Chakilam, A. R., and Torchilin, V. P. (2004) Tumor-targeted liposomes: doxorubicin-loaded long-circulating liposomes modified with anti-cancer antibody. J Control Release 100, 135-44.
Maeda, H., Wu, J., Sawa, T., Matsumura, Y., and Hori, K. (2000) Tumor vascular permeability and the EPR effect in macromolecular therapeutics: a review. J Control Release 65, 271-84.
Maheshwari, A., Mahato, R. I., McGregor, J., Han, S., Samlowski, W. E., Park, J. S., and Kim, S. W. (2000) Soluble biodegradable polymer-based cytokine gene delivery for cancer treatment. Mol Ther 2, 121-30.
Mai, J. C., Shen, H., Watkins, S. C., Cheng, T., and Robbins, P. D. (2002) Efficiency of protein transduction is cell type-dependent and is enhanced by dextran sulfate. J Biol Chem 277, 30208-18.
Palmer, T. N., Caride, V. J., Caldecourt, M. A., Twickler, J., and Abdullah, V. (1984) The mechanism of liposome accumulation in infarction. Biochim Biophys Acta 797, 363-8.
Rudolph, C., Plank, C., Lausier, J., Schillinger, U., Muller, R. H., and Rosenecker, J. (2003) Oligomers of the arginine-rich motif of the HIV-1 TAT protein are capable of transferring plasmid DNA into cells. J Biol Chem 278, 11411-8.
Santra, S., Yang, H., Holloway, P. H., Stanley, J. T., and Mericle, R. A. (2005) Synthesis of water-dispersible fluorescent, radio-opaque, and paramagnetic CdS:Mn/ZnS quantum dots: a multifunctional probe for bioimaging. J Am Chem Soc 127, 1656-7.
Schwarze, S. R., Ho, A., Vocero-Akbani, A., and Dowdy, S. F. (1999) In vivo protein transduction: delivery of a biologically active protein into the mouse. Science 285, 1569-72.
Snyder, E. L., and Dowdy, S. F. (2004) Cell penetrating peptides in drug delivery. Pharm Res 21, 389-93.
Tachibana, R., Harashima, H., Shono, M., Azumano, M., Niwa, M., Futaki, S., and Kiwada, H. (1998) Intracellular Regulation of Macromolecules Using pH-Sensitive Liposomes and Nuclear Localization Signal Qualitative and Quantitative Evaluation of Intracellular Trafficking. Biochemical and Biophysical Research Communications 251, 538-544.
Torchilin, V. P., Levchenko, T. S., Lukyanov, A. N., Khaw, B. A., Klibanov, A. L., Rammohan, R., Samokhin, G. P., and Whiteman, K. R. (2001) p-Nitrophenylcarbonyl-PEG-PE-liposomes: fast and simple attachment of specific ligands, including monoclonal antibodies, to distal ends of PEG chains via p-nitrophenylcarbonyl groups. Biochim Biophys Acta 1511, 397-411.
Torchilin, V. P., Rammohan, R., Weissig, V., and Levchenko, T. S. (2001) TAT peptide on the surface of liposomes affords their efficient intracellular delivery even at low temperature and in the presence of metabolic inhibitors. Proc Natl Acad Sci USA 98, 8786-91.
Torchilin, V. P., Levchenko, T. S., Rammohan, R., Volodina, N., Papahadjopoulos-Sternberg, B., and D'Souza, G. G. (2003) Cell transfection in vitro and in vivo with nontoxic TAT peptide-liposome-DNA complexes. Proc Natl Acad Sci USA 100, 1972-7.
Torchilin, V. P., Lukyanov, A. N., Gao, Z., and Papahadjopoulos-Sternberg, B. (2003) Immunomicelles: targeted pharmaceutical carriers for poorly soluble drugs. Proc Natl Acad Sci USA 100, 6039-44.
Torchilin, V. P. (2004) Targeted polymeric micelles for delivery of poorly soluble drugs. Cell Mol Life Sci 61, 2549-59.
Torchilin, V. P. (2005) Lipid-core micelles for targeted drug delivery. Curr Drug Deliv 2, 319-27.
Torchilin, V. P. (2005) Recent advances with liposomes as pharmaceutical carriers. Nat Rev Drug Discov 4, 145-60.
Vives, E., Brodin, P., and Lebleu, B. (1997) A truncated HIV-1 Tat protein basic domain rapidly translocates through the plasma membrane and accumulates in the cell nucleus. J Biol Chem 272, 16010-7.
Vives, E., Richard, J. P., Rispal, C., and Lebleu, B. (2003) TAT peptide internalization: seeking the mechanism of entry. Curr Protein Pept Sci 4, 125-32.
Wadia, J. S., Stan, R. V., and Dowdy, S. F. (2004) Transducible TAT-HA fusogenic peptide enhances escape of TAT-fusion proteins after lipid raft macropinocytosis. Nat Med 10, 310-5.
Wattiaux, R., Laurent, N., Wattiaux-De Coninck, S., and Jadot, M. (2000) Endosomes, lysosomes: their implication in gene transfer. Advanced Drug Delivery Reviews 41, 201-208.
Widera, A., Norouziyan, F., and Shen, W. C. (2003) Mechanisms of TfR-mediated transcytosis and sorting in epithelial cells and applications toward drug delivery. Adv Drug Deliv Rev 55, 1439-66.
Zhao, M., Kircher, M. F., Josephson, L., and Weissleder, R. (2002) Differential conjugation of tat peptide to superparamagnetic nanoparticles and its effect on cellular uptake. Bioconjug Chem 13, 840-4.
Shen W. C. and Ryser H. J. (1981). cis-Aconityl spacer between daunomycin and macromolecular carriers: a model of pH-sensitive linkage releasing drug from a lysosomotropic conjugate. Biochem. Biophys. Res. Commun. 102(3):1048-54.
Ogden J. R., Leung K., Kunda S. A., Telander M. W., Avner B. P., Liao S. K., Thurman G. B. and Oldham R. K. (1989). Immunoconjugates of doxorubicin and murine antihuman breast carcinoma monoclonal antibodies prepared via an N-hydroxysuccinimide active ester intermediate of cis-aconityl-doxorubicin: preparation and in vitro cytotoxicity. Mol. Biother. 1(3):170-4.
Patel V. F., Hardin J. N., Mastro J. M., Law K. L., Zimmermann J. L., Ehlhardt W. J., Woodland J. M. and Starling J. J. (1996). Novel acid labile COL1 trityl-linked difluoronucleoside immunoconjugates: synthesis, characterization, and biological activity. Bioconjugate Chem. 7(4):497-510.
Heffernan M. J. and Murthy N. (2005). Polyketal nanoparticles: a new pH-sensitive biodegradable drug delivery vehicle. Bioconjugate Chem. 16(6):1340-2.
Gillies E. R. and Frechet J. M. (2005). pH-Responsive copolymer assemblies for controlled release of doxorubicin. Bioconjugate Chem. 16(2):361-8.
Gillies E. R., Jonsson T. B. and Frechet J. M. (2004). Stimuli-responsive supramolecular assemblies of linear-dendritic copolymers. J. Am. Chem. Soc. 126(38):11936-43.
Gumusderelioglu M. and Kesgin D. (2005). Release kinetics of bovine serum albumin from pH-sensitive poly(vinyl ether) based hydrogels. Int. J. Pharm. 288(2):273-9.
Shin J., Shum P. and Thompson D. H. (2003). Acid-triggered release via dePEGylation of DOPE liposomes containing acid-labile vinyl ether PEG-lipids. Journal of Controlled Release 91(1-2):187-200.
Toncheva V., Schacht E., Ng S. Y., Barr J. and Heller J. (2003). Use of block copolymers of poly(ortho esters) and poly (ethylene glycol) micellar carriers as potential tumour targeting systems. J. Drug Target. 11(6):345-53.
Oishi M., Nagasaki Y., Itaka K., Nishiyama N. and Kataoka K. (2005). Lactosylated poly(ethylene glycol)-siRNA conjugate through acid-labile beta-thiopropionate linkage to construct pH-sensitive polyion complex micelles achieving enhanced gene silencing in hepatoma cells. J. Am. Chem. Soc. 127(6):1624-5.
Kong S. D., Luong A., Manorek G., Howell S. B. and Yang J. (2007). Acidic hydrolysis of N-Ethoxybenzylimidazoles (NEBIs): potential applications as pH-sensitive linkers for drug delivery. Bioconjug Chem 18(2):293-6.
Lee E. S., Na K. and Bae Y. H. (2005). Super pH-sensitive multifunctional polymeric micelle. Nano Lett 5(2):325-9.
Sethuraman V. A. and Bae Y. H. (2007). TAT peptide-based micelle system for potential active targeting of anti-cancer agents to acidic solid tumors. J Control Release 118(2):216-24.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A condition-dependent, multiple target delivery system, said delivery system comprising:
a polyfunctional carrier entity;
a first class of targeting functionalities attached to said carrier entity and targeting a target zone; and
a second class of targeting functionalities attached to said carrier entity, wherein said second class of targeting functionalities is shielded when said polyfunctional carrier entity is out of said target zone, but becomes exposed when said polyfunctional carrier entity is inside said target zone.

2. The delivery system of claim 1, wherein said first class of targeting functionalities is not shielded.

3. The delivery system of claim 1, wherein said carrier entity is loaded with a molecule selected from the group consisting of a small molecule drug, a nucleic acid, a diagnostic agent and a research reagent.

4. The delivery system of claim 1, wherein said target zone is in a patient.

5. The delivery system of claim 4, wherein said patient is a human patient.

6. The delivery system of claim 1, wherein said target zone is in cultured tissue.

7. The delivery system of claim 1, wherein said polyfunctional carrier entity is selected from the group consisting of liposomes, micelles, polymeric particles, nanocapsules, niosomes and nanoparticles.

8. The delivery system of claim 4, wherein said target zone in said patient is a tumor site, an infarct site, an infection site or an inflammation site.

9. The delivery system of claim 1, wherein said first class of targeting functionalities comprises an antibody.

10. The delivery system of claim 9, wherein said antibody is cardiac myosin-specific mAb 2G4.

11. The delivery system of claim 1, wherein said first class of targeting functionalities comprises nanoparticles.

12. The delivery system of claim 1, wherein said second class of targeting functionalities is shielded by a shielding construct comprising a first class targeting functionality attached to said carrier entity via a long-chain polymer spacer.

13. The delivery system of claim 12, wherein said first class of targeting functionality is attached to said carrier entity via a condition-dependent bond between said long-chain polymer spacer and said carrier entity.

14. The delivery system of claim 1, wherein said second class of targeting functionalities is shielded by a shielding construct comprising a sterically protective polymer.

15. The delivery system of claim 14, wherein said sterically protective polymer is selected from the group consisting of poly(ethylene glycol), poly(vinyl alcohol) and poly(vinyl propionate).

16. The delivery system of claim 14, wherein said sterically protective polymer is attached to said carrier entity via a condition-dependent bond.

17. The delivery system of claim 13 or claim 16, wherein said condition-dependent bond is cleavable under a condition at said target zone selected from the group consisting of a change in pH, a change in temperature, the presence of a redox agent, a change in oxygen content, enzyme activation, an increase in active oxygen content, an increase in free radical content and hypoxia.

18. The delivery system of claim 1, wherein said second class of targeting functionalities is a specific internalizable ligand.

19. The delivery system of claim 18, wherein said internalizable ligand is folate or transferrin.

20. The delivery system of claim 1, wherein said second class of targeting functionalities is a cell-penetrating peptide.

21. The delivery system of claim 20, wherein said cell-penetrating peptide is TATpeptide or polyarginine.

22. A pharmaceutical composition comprising:
the delivery system of claim 1, wherein said carrier entity in said delivery system is loaded with a pharmaceutical agent.

23. A method of administering a pharmaceutical agent to a patient, said method comprising the steps of:
providing the pharmaceutical composition of claim 22; and
administering to a patient, systemically or locally, an effective amount of said composition.

24. The method of claim 23, wherein said patient is a human patient.
